(19) United States
(12) Patent Application Publication
(10) Pub. No.: US 2021/0009921 A1
(43) Pub. Date: Jan. 14, 2021

(54) PROCESS FOR CONVERTING ATRANOL AND ITS DERIVATIVES INTO HYDROSOLUBLE COMPOUNDS

(71) Applicants: UNIVERSITÉ CÔTE D'AZUR, NICE (FR); CENTRE NATIONAL DE LA RECHERCHE SCIENTIFIQUE, Paris (FR)

(72) Inventors: Sylvain ANTONIOTTI, Villeneuve-Loubet (FR); Hélène BOUGES, Nice (FR); André MONCHOT, Elancourt La Clef St Pierre (FR)

(21) Appl. No.: 16/980,070

(22) PCT Filed: Mar. 12, 2019
(86) PCT No.: PCT/EP2019/056160

§ 371 Date: Sep. 11, 2020

(30) Foreign Application Priority Data
Mar. 12, 2018 (EP) 18305259.6

(51) Int. Cl. C11B 9/02

(52) U.S. Cl. CPC C11B 9/022

(57) ABSTRACT

The present disclosure relates to a process for converting atranol and/or its derivatives into hydrosoluble compound(s). In particular, the present disclosure relates to a process for converting atranol and/or its derivative(s), said process comprising a step of mixing a composition comprising atranol and/or its derivative(s) with an enzyme belonging to the peroxidase family and a peroxide. The present disclosure also relates to a process for producing an oakmoss extract having less than 100 ppm of atranol and/or its derivative(s).

### TECHNICAL FIELD

The present disclosure relates to a process for converting atranol and/or its derivatives into hydrosoluble compounds. In particular, the present disclosure relates to a process for converting atranol and/or its derivative(s), said process comprising a step of mixing a composition comprising atranol and/or its derivative(s) with an enzyme belonging to the peroxidase family and a peroxide.

### BACKGROUND

Complex natural products obtained by extraction techniques are mixtures of dozens of individual compounds from a relatively broad spectrum of chemical families. Although they have been used for thousands of years, they may contain undesirable toxic molecules. The first organ exposed to these molecules when complex natural products are used as ingredients in fragrances and cosmetic products is obviously the skin. As a result, several conditions such as rash, pruritus, blotches and severe eczema, have been attributed to the presence of one or more undesirable toxic molecules. In the particular case of oakmoss extracts, which belong to the premium list of natural ingredients in perfumery, the presence of atranol and chloroatranol has been shown to be responsible for severe skin irritation. As a result, the International Fragrance Association (IFRA) recommends limiting the presence of atranol and chloroatranol to 100 ppm each in oakmoss extracts used in perfumery.

Several methods have been proposed to lower the content of atranol and chloroatranol in oakmoss extracts.

Document EP0468189A2 discloses a process for producing hypoallergenic moss extracts. The moss extract, concrete or absolute, is treated with an amino-acid such as leucine or lysine. The content of different allergenic constituent, including atranol and chloroatranol, is then reduced.

Document WO93/23509 discloses the use of an aldehyde reducing agent to produce hypoallergenic moss extracts. The content of aldehydes, including atranol and chloroatranol, is therefore reduced in the modified moss extract.

However, these methodologies suffer from different drawbacks like lack of selectivity, or waste generation, resulting in an alteration of the olfactory quality of the modified extract or in additional steps of treatment.

Thus, there is a need for a clean method for producing modified oakmoss extracts having the same olfactory quality as the unmodified extracts and a reduced content of atranol and chloroatranol.

The inventors surprisingly found that upon treatment with an enzyme belonging to the peroxidase family and a peroxide, atranol was converted into a hydrosoluble dimeric product of formula (I).

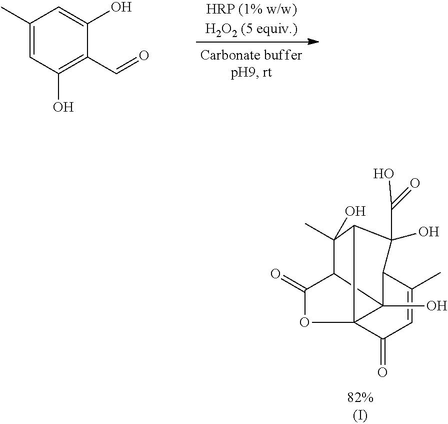 [SMILES: Cc1cc(O)c(C=O)c(O)c1 ; CC1=CC(=O)C23OC(=O)C4C(C)(O)C2C(O)(C(=O)O)C1C43O]

The structure of the dimeric product was determined by ¹H and ¹³C NMR spectroscopy, and in particular COSY, HMBC, and HMQC, and MS (ESI). Acetylation of the dimer further led to a product soluble in organic solvents for which HRMS (QTOF) was performed. This confirmed the proposed structure of the dimeric product.

The inventors also found that this method can be used to produce a modified oakmoss extract with a reduced content of atranol and its derivative chloroatranol, while the olfactory quality of the modified moss extract remained satisfactory.

### SUMMARY

Accordingly, the present disclosure relates first to a process for converting atranol and/or its derivative(s) into hydrosoluble compound(s), said process comprising a step of mixing a composition comprising atranol and/or its derivative(s) with an enzyme belonging to the peroxidase family and a peroxide.

The disclosure also relates to a process for producing an oakmoss extract having less than 100 ppm of atranol and/or its derivative(s), said process comprising the following steps:
a) mixing an oakmoss extract comprising atranol and/or its derivative(s) with an enzyme belonging to the peroxidase family and a peroxide,
b) incubating the mixture, to convert atranol and/or its derivative(s) into hydrosoluble compound(s),
c) eliminating the hydrosoluble compound(s) using liquid/liquid extraction.

The disclosure also relates to the use of an enzyme belonging to the peroxidase family and a peroxide for reducing the content of atranol and/or its derivative(s) from a moss extract.

### DETAILED DESCRIPTION

Various embodiments of the disclosure are described herein. It will be recognized that features specified in each embodiment may be combined with other specified features to provide further embodiments.

Process for Converting Atranol and/or its Derivative(s) into Hydrosoluble Compound(s)

The present disclosure relates first to a process for converting atranol and/or its derivative(s) into hydrosoluble compound(s), said process comprising a step of mixing a composition comprising atranol and/or its derivative(s) with an enzyme belonging to the peroxidase family and a peroxide.

As used herein, the terms “atranol and/or its derivative(s)” refer to atranol and compounds which are derived from atranol by replacement of one atom with another atom or group of atoms.

According to an embodiment, the derivatives of atranol are chloroatranol, ethyl hematommate, ethyl chlorohematommate, atranorin and chloroatranorin, as represented below. Thus, in this embodiment, the terms “atranol and/or its derivative(s)” refer to compounds selected from atranol, chloroatranol, ethyl hematommate, ethyl chlorohematommate, atranorin, chloroatranorin, and mixtures thereof.

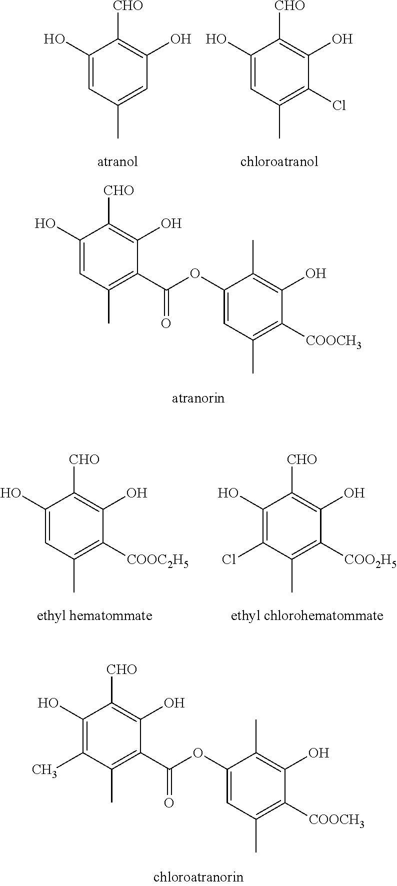

Preferably, the terms “atranol and/or its derivative(s)” refer to compounds selected from atranol, chloroatranol, and mixtures thereof.

As used herein, the term “hydrosoluble compound” refers to a compound which is soluble in water, for example, at a concentration of at least 0.1 g/100 mL of water at 20° C.

According to an embodiment of the process, atranol and/or its derivative(s) are converted into hydrosoluble dimer(s). According to an embodiment, atranol is converted into a hydrosoluble dimer of formula (I)

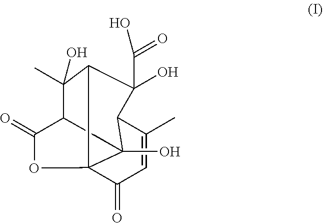

Advantageously, the composition comprising atranol and/or its derivative(s) is a moss extract, preferably a moss oil, concrete or absolute. The moss extracts useful in the present disclosure are generally the ones obtained by solvent extraction of lichens and include in particular the oakmoss concrete and absolute (from Evernia prunastri L.). Preferably, the composition comprising atranol and/or its derivative(s) is an oakmoss extract. According to an embodiment, the composition comprising atranol and/or its derivative(s) is an oakmoss concrete. According to another embodiment, the composition comprising atranol and/or its derivative(s) is an oakmoss absolute.

As used herein, the terms “enzyme belonging to the peroxidase family” refers to an enzyme having the ability to catalyse the oxidation of an organic substrate using a peroxide as terminal oxidant, the enzyme being obtained from a wild or mutant living organism.

According to an embodiment, the enzyme belonging to the peroxidase family is selected from horseradish peroxidase (HRP), soybean peroxidase (SPB), myeloperoxidase (MPO), lactoperoxidase (LPO), cytochrome C peroxidase (DiHCcP), and mixtures thereof.

According to a preferred embodiment, the enzyme belonging to the peroxidase family is HRP.

The amount of enzyme belonging to the peroxidase family can be at least 0.1% by weight compared to the weight of atranol and/or its derivatives, preferably at least 0.5%, and more preferably at least 1%. According to an embodiment, the amount of enzyme belonging to the peroxidase family is comprised between 0.1% and 25% by weight compared to the weight of atranol and/or its derivatives, or between 1% and 15%.

According to an embodiment, the peroxide used in the process is chosen among hydrogen peroxide (H₂O₂), hydroperoxides (RO₂H), organic peroxides (RO₂R′) and mixtures thereof. As used herein, the term “hydroperoxides” refer to compounds having the skeleton ROOH, in which R is an organyl group, preferably R is a linear or branched alkyl group, an acyl group such as acetyl or benzoyl, or an aryl group. Advantageously, R comprises between 1 and 18 carbon atoms. Hydroperoxides include peroxy acids like peroxyacetic acid, peroxybenzoic acid, and meta-chloroperoxybenzoic acid. As used herein, the terms “organic peroxides” refer to compounds having the skeleton ROOR′, in which R and R′ are an organyl group, preferably R and R′ are, independently, linear or branched alkyl groups, acyl groups such as acetyl or benzoyl, or aryl groups. Advantageously, R and R′ comprise, independently, between 1 and 18 carbon atoms.

Preferably, the peroxide is H₂O₂.

The amount of peroxide can be at least 0.5 molar equivalent compared to atranol and/or its derivatives, preferably at least 1 molar equivalent, and more preferably at least 2 molar equivalents. According to an embodiment, the amount of peroxide is comprised between 1 and 5 molar equivalents compared to atranol and/or its derivatives, or between 1.5 and 3 molar equivalents.

According to an embodiment, the mixing step lasts at least 0.5 hour, preferably at least 1 hour, and more preferably at least 2 hours. According to an embodiment, the mixing step lasts between 1 and 10 hours, or between 2 and 4 hours.

The process can be carried out in an aqueous solution. Preferably, the process is carried out in an aqueous buffer solution, like a carbonate buffer.

According to an embodiment, the process is carried out at basic pH, for example at a pH between 8 and 10.

According to an embodiment, the present disclosure relates to a process for converting atranol and/or chloroatranol into hydrosoluble compound(s), said process comprising a step of mixing a moss extract comprising atranol and/or chloroatranol with HRP and H₂O₂.

According to an embodiment, the present disclosure relates to a process for converting atranol and/or chloroatranol into hydrosoluble compound(s), said process comprising a step of mixing oakmoss concrete or absolute comprising atranol and/or chloroatranol with HRP and H₂O₂, wherein the amount of H₂O₂ is at least 2 molar equivalents compared to atranol and/or chloroatranol, the amount of HRP is at least 1% by weight compared to the weight of atranol and/or its derivatives, and the mixing step lasts at least 2 hours.

The present disclosure also relates to a process for converting atranol into a hydrosoluble dimer of formula (I)

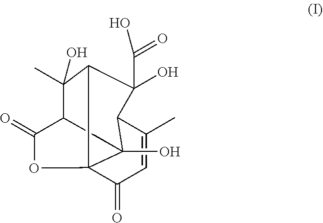

said process comprising a step of mixing a composition comprising atranol, preferably a moss extract, with an enzyme belonging to the peroxidase family, preferably HRP, and a peroxide, preferably H₂O₂.

Process for Producing a Modified Oakmoss Extract

The disclosure also relates to a process for producing an oakmoss extract having less than 100 ppm of atranol and/or its derivative(s), said process comprising the following steps:
a) mixing an oakmoss extract comprising atranol and/or its derivative(s) with an enzyme belonging to the peroxidase family and a peroxide,
b) incubating the mixture, to convert atranol and/or its derivative(s) into hydrosoluble compound(s),
c) eliminating the hydrosoluble compound(s) using liquid/liquid extraction.

During steps a) and b), atranol and/or its derivative(s) are converted into hydrosoluble compound(s) which can be eliminated using liquid/liquid extraction. Advantageously, the liquid/liquid extraction of step c) is performed using an aqueous solution and an organic solvent. In this case, the modified oakmoss extract is recovered in the organic solvent, while the hydrosoluble compound(s), into which atranol and/or its derivatives are converted, are discarded with the aqueous phase. The obtained modified oakmoss extract is an oakmoss extract having less than 100 ppm of atranol and/or its derivative(s).

Preferably, the oakmoss extract is an oakmoss concrete or absolute.

According to an embodiment, the enzyme belonging to the peroxidase family is selected from horseradish peroxidase (HRP), soybean peroxidase (SPB), myeloperoxidase (MPO), lactoperoxidase (LPO), cytochrome C peroxidase (DiHCcP), and mixtures thereof.

According to a preferred embodiment, the enzyme belonging to the peroxidase family is HRP.

The amount of enzyme belonging to the peroxidase family can be at least 0.1% by weight compared to the weight of atranol and/or its derivatives, preferably at least 0.5%, and more preferably at least 1%. According to an embodiment, the amount of enzyme belonging to the peroxidase family is comprised between 0.1% and 25% by weight compared to the weight of atranol and/or its derivatives, or between 1% and 15%.

According to an embodiment, the peroxide used in the process is chosen among hydrogen peroxide (H₂O₂), hydroperoxides (RO₂H), organic peroxides (RO₂R′) and mixtures thereof. As used herein, the term “hydroperoxides” refer to compounds having the skeleton ROOH, in which R is an organyl group preferably R is a linear or branched alkyl group, an acyl group such as acetyl or benzoyl, or an aryl group. Advantageously, R comprises between 1 and 18 carbon atoms. Hydroperoxides include peroxy acids like peroxyacetic acid, peroxybenzoic acid, and meta-chloroperoxybenzoic acid. As used herein, the terms “organic peroxides” refer to compounds having the skeleton ROOR′, in which R and R′ are an organyl group, preferably R and R′ are, independently, linear or branched alkyl groups, acyl groups such as acetyl or benzoyl, or aryl groups. Advantageously, R and R′ comprise, independently, between 1 and 18 carbon atoms. Preferably, the peroxide is H₂O₂.

The amount of peroxide can be at least 0.5 molar equivalent compared to atranol and/or its derivatives, preferably at least 1 molar equivalent, and more preferably at least 2 molar equivalents. According to an embodiment, the amount of peroxide is comprised between 1 and 5 molar equivalents compared to atranol and/or its derivatives, or between 1.5 and 3 molar equivalents.

Steps a) and b) can be carried out in an aqueous solution. Preferably, steps a) and b) are carried out in an aqueous buffer solution, like a carbonate buffer.

According to an embodiment, steps a) and b) are carried out at basic pH, for example at a pH between 8 and 10.

According to an embodiment, step b) lasts at least 0.5 hour, preferably at least 1 hour, and more preferably at least 2 hours. According to an embodiment, step b) lasts between 1 and 10 hours, or between 2 and 4 hours.

Advantageously, when the liquid/liquid extraction of step c) is performed using an aqueous solution and an organic solvent, the organic solvent is chosen among common organic solvent like ether oxides, hydrocarbons or ethyl acetate.

The process can also comprise a step d) of recovering the oakmoss extract having less than 100 ppm of atranol and/or its derivative(s) in an organic solvent.

According to an embodiment, the modified oakmoss extract show no difference in terms of olfactory properties with the initial oakmoss concrete or absolute.

According to an embodiment, the disclosure also relates to a process for producing an oakmoss concrete or absolute having less than 100 ppm of atranol and/or chloroatranol, said process comprising the following steps:
a) mixing an oakmoss concrete or absolute comprising atranol and/or chloroatranol with HRP and H₂O₂, wherein the amount of H₂O₂ is at least 2 molar equivalents compared to atranol and/or chloroatranol, and the amount of HRP is at least 1% by weight compared to the weight of atranol and/or chloroatranol,
b) incubating the mixture for at least 2 hours, to convert atranol and/or its derivative(s) into hydrosoluble compound(s),
c) eliminating the hydrosoluble compound(s) using an aqueous solution,
d) recovering the oakmoss concrete or absolute having less than 100 ppm of atranol and/or its derivative(s) in an organic solvent.

According to an embodiment, the disclosure also relates to a process for producing an oakmoss extract having less than 100 ppm of atranol, said process comprising the following steps:
a) mixing an oakmoss extract comprising atranol with an enzyme belonging to the peroxidase family and a peroxide,
b) incubating the mixture, to convert atranol into a hydrosoluble dimer of formula (I)

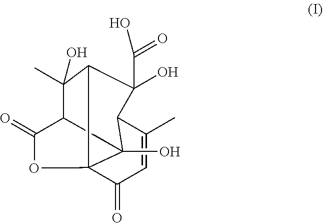

c) eliminating the hydrosoluble dimer of formula (I) using liquid/liquid extraction.

The disclosure also relates to the use of an enzyme belonging to the peroxidase family, preferably HRP, and a peroxide, preferably H₂O₂, for reducing the content of atranol and/or its derivative(s) from a moss extract. Preferably, the moss extract is an oakmoss concrete or absolute.

### FIGURES LEGENDS

FIG. 1 shows the HPLC-PDA chromatograms of an oakmoss absolute before (top) and after (bottom) treatment according to example 4 (2 equivalents of H₂O₂ and 2 hours reaction time). Insets are zoom of the area of the chromatogram where atranol and chloroatranol are eluting (respectively at 16.9 and 20.8 min).

### EXAMPLES

### Example 1: Conversion of Pure Atranol

Atranol (0.66 mmol) was dissolved in pH9 carbonate buffer 20 mM at room temperature to reach a concentration of 2 g/L. HRP was then introduced at a 1% wt ratio and the reaction was started with the slow addition of 2 equiv. of H₂O₂ (30% w/w aqueous solution) at a 0.1 mL/h flow rate. After 4 hours, the aqueous phase was extracted with AcOEt, and both the organic and aqueous phases were evaporated and analyzed. The same protocol was used to perform control experiments. The different conditions and results are summarized in table 1.

TABLE 1
Conversion of pure atranol

| | | | Product in | Product in |
|---|---|---|---|---|
| | | | the organic | the aqueous |
| Entry | H₂O₂ | HRP | phase (yield) | phase (yield) |
| 1 | — | — | Atranol (95%)^{a} | — |
| 2 | — | 1% (w/w) | Atranol (92%)^{a} | — |
| 3 | 2 eq. | — | 5-methylpyrogallol (84%) | — |
| 4 | 2 eq. | 1% (w/w) | 5-methylpyrogallol (2%) | Dimer (75%) |

^{a}Recovery of starting material.

In the absence of H₂O₂, atranol was recovered unchanged upon extraction with AcOEt either with or without HRP used in 1% w/w (table 1, entries 1 and 2). In the presence of H₂O₂ but without HRP, 5-methylpyrogallol was obtained in 84% yield upon extraction with AcOEt (table 1, entry 3). Surprisingly, in the presence of HRP (1% w/w) and H₂O₂ (2 equiv.), only 2% of 5-methylpyrogallol were isolated by extraction with AcOEt. Upon evaporation of the aqueous phase, a dimer was obtained in 75% yield.

### Example 2: HRP-Catalyzed Oxidation of Pure Atranol

HRP (124 U/mg, 4 mg) was dissolved in 65 mL of pH9 carbonate buffer (20 mM) containing atranol (200 mg, 1.32 mmol). The reaction flask was covered with an aluminum foil to avoid peroxide decomposition. Reaction was initiated by the slow addition of a 30% aqueous H₂O₂ solution at 0.1 mL/h to ensure the final addition of 2 equivalents of hydrogen peroxide (0.264 mL) with respect to atranol. After 6 hours at room temperature, an aqueous HCl solution (0.1 M) was added until pH4 is reached. This aqueous layer was extracted with ethyl acetate (3×70 mL). The aqueous phases were concentrated by rotary evaporation to give the dimer as a white powder (193 mg, 44%). ESI-MS: m/z=310. ¹H RMN (D₂O, 400 MHz): δ ppm 6.37 (t, 1H), 3.97 (d, 1H), 3.37 (s, H), 2.68 (s, 2H), 2.62 (s, H), 2.14 (s, 3H), 1.79 (s, 3H) ¹³C RMN: (D₂O, 100 MHz) δ ppm 197.5 (C), 178.1 (C), 172.3 (C), 164.3 (C), 127.1 (CH), 88.9 (C), 87.1 (C), 85.7 (C), 77.7 (C), 60.2 (CH), 54.9 (CH), 52.9 (CH), 25.7 (CH₃), 23.7 (CH₃).

### Example 3: Acetylation of the Dimer

Since the dimer could not be analyzed directly by GC-MS, its acetylated derivative was prepared. Dimer (0,100 g, 0.32 mmol) was dissolved in distilled CH₂Cl₂ (2 ml). Triethylamine was then added (0.28 ml, 2.1 mmol) followed by Ac₂O (0.2 ml; 2.1 mmol) under an inert atmosphere. After completion of the reaction, the crude reaction mixture was evaporated in vacuo. The obtained solid was dissolved in CH₂Cl₂ and washed with water. After drying of the organic layer with MgSO₄ and evaporation, the triacetylated dimer was obtained as an oil (0.12 g, 90%). HRMS: C₂₀H₁₉O₁₀ for [MH⁺], calc. 419.0978; found 419.0950, Δ=6.6 ppm.

### Example 4: HRP-Catalyzed Removal of Atranol (4.3%) and Chloroatranol (2.3%) from Oakmoss Absolute at the Milligram Scale

Oakmoss absolute (250 mg) containing atranol (4.3%) and chloroatranol (2.3%) was dissolved in 270 mL of pH9 carbonate buffer (20 mM). After sonication of the reaction mixture during 2 hours, HRP (124 U/mg, 2 mg) was added. The reaction flask was covered with an aluminum foil, and 1, 2, 3 or 4 equivalents of 30% aqueous H₂O₂ solution were added. The agitation was maintained for 2 or 4 hours at room temperature. Extraction by ethyl acetate allowed the recovery of the modified absolute (231 mg, 92%, with 2 equivalents of H₂O₂ and 4 hours reaction time) after drying over magnesium sulfate, filtration and solvent removal by rotary evaporation. HPLC-UV-MS and GC-MS analysis of the modified oakmoss absolute confirmed the disappearance of atranol and chloroatranol. HPLC with UV detection was not suitable to quantify trace amounts of atranol and in this regard, MS detection was used with an external calibration method (S. C. Rastogi, R. Bossi, J. D. Johansen, T. Menné, G. Bernard, E. Giménez-Arnau and J.-P. Lepoittevin, Content of oak moss allergens atranol and chloroatranol in perfumes and similar products Contact Dermatitis 2004 50, p. 367-370, R. Bossi, S. C. Rastogi, G. Bernard, E. Gimenez-Arnau, J. D. Johansen, J.-P. Lepoittevin and T. Menné A liquid chromatography-mass spectrometric method for the determination of oak moss allergens atranol and chloroatranol in perfumes Journal of Separation Science 2004 27, p. 537-540). The results are presented in table 2.

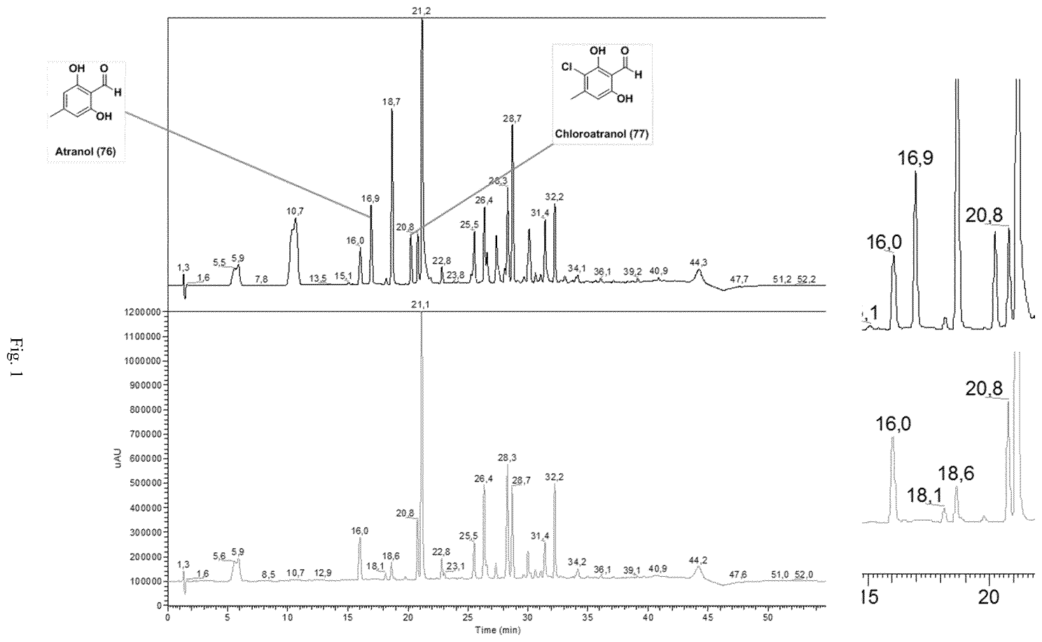

To assess the overall effect on the entire oakmoss absolute, HPLC-UV was used. The chromatograms of the starting oakmoss absolute and the obtained modified oakmoss absolute are shown in FIG. 1. They show that chemical composition of the absolute remained broadly the same.

TABLE 2
Atranol content of the modified oakmoss
absolute after enzymatic reaction

| | | | Atranol title |
|---|---|---|---|
| Entry | H₂O₂ | Reaction time | (ppm) |
| 1 | 1 equiv. | 2 h | 141 |
| 2 | 2 equiv. | 2 h | 65 |
| 3 | 3 equiv. | 2 h | 32 |
| 4 | 4 equiv. | 2 h | 12 |
| 5 | 1 equiv. | 4 h | 130 |
| 6 | 2 equiv. | 4 h | 62 |
| 7 | 3 equiv. | 4 h | 28 |
| 8 | 4 equiv. | 4 h | 7 |

Residual values of atranol below 100 ppm were obtained when more than 2 equivalents of H₂O₂ were used. With 4 equivalents of H₂O₂ and upon 4 hours of reaction, a residual title as low as 7 ppm of atranol was observed.

### Example 5: HRP-Catalyzed Removal of Atranol (4.3%) and Chloroatranol (2.3%) from Oakmoss Absolute at the Gram Scale

The oakmoss absolute (1.2 g) was dissolved in 1.3 L of pH9 carbonate buffer (20 mM). After sonication of the reaction mixture during 2 hours, HRP (124 U/mg, 10 mg) was added. The reaction flask was covered with an aluminum foil, and 2 equivalents of 30% aqueous H₂O₂ solution (97 μl) were added. The agitation was maintained 2 hours at room temperature. Extraction by ethyl acetate allowed the recovery of the modified absolute (1.1 g, 91%) after drying over magnesium sulfate, filtration and solvent removal by rotary evaporation. The modified oakmoss absolute contained 60 ppm of atranol and chloroatranol was not detected (HPLC-MS, SIM mode). HPLC-UV-MS and GC-MS analysis confirmed this result.

### Example 6: Sensory Analysis

The conserved olfactory quality of the modified oakmoss absolute was assessed by sensory analysis following the triangular testing methodology. Three identical vials containing 2 samples of oakmoss absolute and 1 sample of modified oakmoss absolute, as solution in EtOH (0.5% w/w), were submitted to a panel of 56 persons taken separately which were asked to identify the modified sample. The following formulae were used, with n₁₋₃ being the value one should exceed to be sure that the result is not statistical distribution for a given level of confidence and N the number of panelists:

$$For 95 \% of confidence \text{:} n 1 = 0.77 \sqrt{N} + \frac{2 N + 3}{6} + 0.5$$

$$For 98 \% of confidence \text{:} n 2 = 1.10 \sqrt{N} + \frac{2 N + 3}{6} + 0.6$$

$$For 9.99 \% of confidence \text{:} n 3 = 1.46 \sqrt{N} + \frac{2 N + 3}{6} + 0.8$$

For a panel of 56 persons, n₁=25.9, n₂=28.0, and n₃=30.9.

As a result, the scores obtained were 18, 16 and 22, respectively. A score of 22 means that, even at the lowest level of confidence, the panel was far from being able to distinguish the modified sample.

1. Process for converting atranol and/or its derivative(s) into hydrosoluble compound(s), said process comprising a step of mixing a composition comprising atranol and/or its derivative(s) with an enzyme belonging to the peroxidase family and a peroxide.

2. Process according to claim 1, wherein the composition comprising atranol and/or its derivative(s) is a moss extract.

3. Process according to claim 1, wherein the composition comprising atranol and/or its derivative(s) is an oakmoss concrete.

4. Process according to claim 1, wherein the composition comprising atranol and/or its derivative(s) is an oakmoss absolute.

5. Process according to claim 1, wherein the enzyme is horseradish peroxidase (HRP).

6. Process according to claim 1, wherein the peroxide is H₂O₂.

7. Process according to claim 6, wherein the amount of peroxide is at least 0.5 molar equivalent compared to atranol and/or its derivatives.

8. Process according to claim 1, wherein the amount of enzyme belonging to the peroxidase family is at least 0.1% by weight compared to the weight of atranol and/or its derivatives.

9. Process according to claim 1, wherein the mixing step lasts at least 0.5 hour.

10. Process according to claim 1, wherein the process is carried out in an aqueous buffer solution.

11. Process according to claim 1, wherein the process is carried out at a pH comprised between 8 and 10.

12. Process according to claim 1, wherein atranol is converted into a hydrosoluble dimer of formula (I)

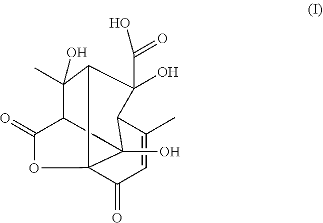

13. Process for producing an oakmoss extract having less than 100 ppm of atranol and/or its derivative(s), said process comprising the following steps:
a) mixing an oakmoss extract comprising atranol and/or its derivative(s) with an enzyme belonging to the peroxidase family and a peroxide,
b) incubating the mixture, to convert atranol and/or its derivative(s) into hydrosoluble compound(s),
c) eliminating the hydrosoluble compound(s) using liquid/liquid extraction.

14. (canceled)

15. Process according to claim 7, wherein the amount of peroxide is at least 1 molar equivalent

16. Process according to claim 7, wherein the amount of peroxide is at least 2 molar equivalents.

17. Process according to claim 8, wherein the amount of enzyme belonging to the peroxidase family is at least 0.5% compared to the weight of atranol and/or its derivatives.

18. Process according to claim 8, wherein the amount of enzyme belonging to the peroxidase family is at least 1% compared to the weight of atranol and/or its derivatives.

19. Process according to claim 1, wherein the mixing step lasts at least 0.5 hour.

20. Process according to claim 1, wherein the mixing step lasts at least 1 hour.

21. Process according to claim 1, wherein the mixing step lasts at least 2 hours.
